(19) United States
(12) Patent Application Publication
(10) Pub. No.: US 2023/0226320 A1
(43) Pub. Date: Jul. 20, 2023

(54) METHOD OF MAKING LARGE NEEDLE INSERTION INTO AN ARTERY OR VEIN EASIER AND WITH LESS UNNECESSARY TRAUMA TO THE BLOOD VESSEL AND METHODS FOR MANUFACTURING SAME (NISE)

(71) Applicant: HemoCleanse Technologies LLC, Lafayette, IN (US)

(72) Inventors: STEPHEN ASH, Lafayette, IN (US); THOMAS SULLIVAN, Lafayette, IN (US)

(73) Assignee: HemoCleanse Technologies LLC, Lafayette, IN (US)

(21) Appl. No.: 18/190,182

(22) Filed: Mar. 27, 2023

Related U.S. Application Data
(63) Continuation-in-part of application No. 17/861,959, filed on Jul. 11, 2022.
(60) Provisional application No. 63/219,946, filed on Jul. 9, 2021.

(51) Int. Cl. A61M 25/06; A61M 5/32

(52) U.S. Cl. CPC A61M 25/06; A61M 5/32; A61M 5/158

(57) ABSTRACT

A method and devices for making needle insertion safer and easier (NISE). NISE or CISE device includes a small needle, cannula, and large needle in a nested assembly to provide less trauma to a target vessel during large needle insertion. The small needle is movable and within the cannula to a position exterior the cannula. The cannula is movable within the large needle. In a preferred assembly, the cannula is provided with a dulled tip having a beak and successive cutting edges along a tapered shape. The device can be provided in a stand-alone device or alternately integrated into existing off the shelf and currently available vascular devices.

### CROSS REFERENCE TO RELATED APPLICATIONS

This U.S. Patent Application in a continuation in part of U.S. patent application Ser. No. 17/861959 filed Jul. 11, 2022 claiming priority to U.S. Provisional Patent Application No. 63/219946 filed 9 Jul. 2021 to the above-named inventors of which the disclosure is considered part of the disclosure of this application and is herein incorporated by reference in its entirety.

### FEDERALLY SPONSORED RESEARCH OR DEVELOPMENT

Not Applicable

### SEQUENCE LISTING, A TABLE, OR A COMPUTER PROGRAM

Not Applicable

### FIELD OF THE INVENTION

This invention relates generally to large needles and a large needle assembly and method for use and manufacturing. More particularly, embodiments of the invention provide a nested assembly of a small needle, cannula, and large needle in an aligned assembly for receipt in a support body. Further, the invention relates generally a large needle assembly, a method of manufacture and use for making large needle insertion into an artery or vein easier and with less unnecessary trauma to the blood vessel.

BACKGROUND

It is often medically necessary to gain access to a vessel using a large needle, the outside diameter of which may be a substantial fraction of the diameter of the vessel. This requires accurate placement of the large needle to avoid unnecessary trauma to the vessel. Some vessels may have thick or tough walls making insertion difficult.

For insertion of a large needle or catheter into a vessel, the tip of the needle or catheter must be sharp to puncture the surrounding tissues and vessel wall. However, for stability and surety that the needle or catheter stays in the vessel and in the proper position, the needle or catheter must be long enough to be advanced into the vessel a distance much greater than the diameter of the vessel. This can be done with a needle, but with the tip of a needle being very sharp, advancing it in any direction may place the tip in contact with the vessel wall resulting in potential damage to the delicate vessel lining. Therefore, the person placing the needle must be able to sense when the tip enters the vessel and then lower the angle of insertion so that the needle tip stays central within the vessel while the needle is advanced. This takes considerable experience and skill but is still subject to chance and potential vessel damage due to normal variability in anatomy from patient to patient and in patients over time, change of medical condition.

One solution within the art to this problem, is the use of a blunt tipped plastic cannula with an internal sharp needle that is used to enter the vessel. This is the standard peripheral IV cannula. After the needle and catheter have entered the vessel, the needle can be retracted so that the blunt-tipped catheter is next advanced into the vessel. This works reasonably well but inserting the catheter portion requires more force than inserting the needle and deforms the vessel. Often the needle enters the vessel properly, but the catheter does not. The catheter will not then advance to its correct position within the vessel.

For the specific medical procedure of kidney dialysis, accesses are typically surgically arterialized vessels (fistulas) or plastic tubes (grafts) which have a very thick and tough wall, making the insertion of needles sometimes difficult. Large catheters with internal needles are even more difficult to insert, due to the lack of a cutting surface on the catheter. Accordingly, using what is called a fistula needle provides better blood flow at less pressure drop, because the needle walls are much thinner than the walls of plastic catheters. The catheters suited for fistulas and dialysis access are also considerably more expensive than fistula needles. For all these reasons, needles are inserted into fistulas and grafts routinely, not catheters. Often the points of these needles penetrate the other side of the accessed vessel, near to the entry site. Especially when the interior of the access is irregular in shape or has stenosis, it is difficult to advance the needle within the fistulas or grafts, because the tip impinges on the inside wall of the access. Invariably each point of contact of the needle point will cause damage to the delicate intimal layer of the interior surface of the accessed vessel.

Another solution to having a sharp entry point but then a blunt object for advancing within the vessel interior is the “Seldinger” technique. For this technique, a sharp needle is inserted in the vessel, and then a blunt “guidewire” is inserted through the needle and advanced into the vessel. The needle is then removed and the guidewire is advanced, usually under fluoroscopic imaging in the radiology department. A dilator with a surrounding catheter is advanced over the guidewire, and the dilator and guidewire are then removed, and the catheter is ready for use. Everything used in the technique must be sterile and protected from contamination. The person placing the device and assistants must don sterile gloves and gowns, and a wide sterile surgical field must be created around the insertion site to the vessel. The guidewires are usually long and flexible, so maintaining their sterility requires attention to the varying course of the guidewire. This technique works well in a radiology department but is not practical for dialysis units or stand-alone dialysis centers.

BRIEF SUMMARY OF THE INVENTION

The present invention in the form of an improved large needle assembly and method for use, is generally referred to herein by the acronym N.I.S.E or NISE standing for: Needle Insertion Safer and Easier (“NISE”) or C.I.S.E. or CISE standing for a combination of an existing ClampCath product with NISE features (“CISE”). ClampCath is what is commonly referred to as the Clampcath SP 502 or Supercath or SupercathNEO produced by Togo Medikit Co. Ltd. This Clampcath (“CC”) device generally resembles a typical IV catheter with the exception of a flexible translucent portion that can be squeezed during insertion. Although not specifically described herein, it is also likely that the NISE or CISE device 10 modifications to the ClampCath product may be incorporated into existing and other IV style catheters without departing from the spirit of this inventive disclosure with the small addition of a translucent area to determine proper vessel entry.

The NISE device and method for use is intended to alleviate the disadvantages of the prior art by realizing at least three concepts: The first concept is the use of blunted points on penetrators other than the first penetrator, of which the first penetrator is retracted into a cylindrical cannula after initial vessel entry. The second is that the cannula has increasing diameters and cutting surfaces to enlarge the entry hole as it is advanced into the vessel. The third is that the access needle or catheter are carried into the vessel while surrounding the cannula. The cannula and the sharp needle (retracted into the cannula) are removed, leaving the access needle or catheter within the access, ready for use.

For better understanding, it may be best to first explain the method of use of the NISE device relative to the inventive structures and features, which will be discussed in an additional detail. In use of the device for the insertion of a large needle into a vessel, the first step is the entry of a small needle into the vessel, allowing the operator to verify entry into the vessel by blood emission or sight of blood at the NISE device. This small needle is sharp for easy initial entry. If this needle is wrongly inserted, for example, by hitting a vessel wall, trauma is minimal due to the small size of this first small needle.

Second, a larger cylindrical cannula, with the larger cylindrical cannula surrounding the small needle and coaxial with the small needle received within an aperture of the larger cylindrical cannula, is advanced with the needle a short distance into the target vessel. The position of the larger cylindrical cannula is held constant while the small needle is retracted (preferably done by spring action, controlled by the user) within a body of the device. The larger cylindrical cannula has a relatively dull tip and typically sharp outer cutting edges near or adjacent the tip. The large cylindrical cannula has an expanding size and cutting surfaces to enlarge the hole in the vessel as the large cylindrical cannula is advanced into the vessel. As the larger cylindrical cannula is advanced into the vessel, if contact is made with the opposite or an adjacent vessel wall, the dull tip of the large cylindrical cannula will minimize potential trauma to the vessel interior walls.

Third, when the large cylindrical cannula is fully advanced into the vessel, a large needle or catheter, referred to herein as an access needle and surrounding a posterior portion of the large cylindrical cannula, is brought into the vessel. If the large cylindrical cannula was advanced a full portion of a length of the large cylindrical cannula into the vessel, and the access needle is completely inserted in the vein, then the access needle is held in place and the cannula and enclosed small needle are removed from the device. If, however, the tip of the cannula met some obstruction during advancement in the vessel and the access needle was only partly inserted into the fistula, then the large cylindrical cannula can be held in place while the access needle is advanced over it.

Fourth, on removal of the large cylindrical cannula and small needle from the access needle interior, a tip of the small needle is drawn through an external tubing segment, elastomeric seal, or valve which automatically seals against leaks. The tubing which is attached to the access needle is then ready for blood removal or fluid infusion during the medical procedure.

In summary, the device includes three separate components which enter the vessel successively: the small sharp needle which first enters the vessel, the large cylindrical cannula having a tip surrounding the small sharp needle and having enlarging diameters and/cutting surfaces along its length (going outwards along the length from the cannula tip), and the large access needle surrounding the outer part of the large cylindrical cannula.

In operation, the sharp small needle is retracted into the large cylindrical cannula tip after insertion of both to the vessel. The large cylindrical cannula and access needle are then advanced into the vessel, and the cannula and small needle are then removed when the access needle is inserted to the proper position within the vessel.

These structures and their functions are all preferably contained in a single integrated unit, part of which may consist of slightly modified presently available vessel access products. Other features and capabilities may be added as described in the detailed description of the embodiments of this disclosure.

In an improvement of the proposed NISE assembly, the CISE device is constructed to provide an improved concentric nested assembly, wherein the successive points of the device are arranged in a more adjacent position when compared to a standard concentric arrangement (FIG. 13). In a method of assembly, the outer tube of the CISE device may be crimped in at least one place (preferably two places) to provide the improved concentric configuration and provide a minimal amount of friction during use. In addition to a crimped assembly method, the CISE device is provided with an improved cannula tip and shape to reduce insertion force.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 shows a schematic of the basic stages of NISE or CISE device insertion from top to bottom relative to FIG. 1, according to the present disclosure;

FIG. 2 is a top view of an exemplary NISE device, according to the present disclosure;

FIG. 3 shows a sideview of the CISE device, according to the present disclosure;

FIG. 4 shows a magnified view of the cannula tip of an exemplary CISE device showing the various preferred angles of an exemplary cannula tip, according to the present disclosure;

FIG. 5 shows a side view of an exemplary modified cannula tip of the CISE device, according to the present disclosure;

FIG. 6 shows a side view of the exemplary modified cannula tip of the CISE device device with a small needle in an extended position according to the present disclosure;

FIG. 7 shows a side view of the small needle release mechanism of the CISE device, according to the present disclosure;

FIG. 8 shows a partial view of the small needle release mechanism of the CISE device with the small needle in a retracted position, according to the present disclosure;

FIG. 9 shows a side view of the CISE assembly, according to the present disclosure;

FIG. 10 shows the side view of the CISE assembly of FIG. 9 with the small needle retracted showing the ability of the cannula to freely move, according to the present disclosure;

FIG. 11 shows a side view of a crimped needle assembly to provide an improved concentric needle assembly, according to the present disclosure;

FIG. 12 shows a side view of an exemplary jig assembly related to a method for crimping a larger tube for aligning a smaller needle within the large tube to facilitate the improved concentric needle assembly, according to the present disclosure; and

FIG. 13 shows an end view of an improved concentric needle assembly, according to the present disclosure.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

Within the specification the following specific definitions or terms may be used:

NISE: Needle Insertion Safer and Easier, an acronym used in this patent application to indicate the devices, their embodiments, methods of manufacture, and methods of use covered under this application.

CISE: A NISE device as defined above with an assembly utilizing the available ClampCath needle assembly that is commonly known within the hemodialysis industry.

Vessel: Either an artery or vein, human or animal, natural, graft, artificial, or some combination thereof.

Cutting Edge: A means by which to separate tissue, for example, by incision or blunt dissection, to facilitate entry into a vessel.

Penetrator: A needle, cannula, or similar tubular (but not necessarily circular) structure that includes one or more cutting edges. It may contain various extra holes or other features known to those skilled in the art.

Needle: A penetrator of some kind whose overall shape would be visually recognized as a “needle” by clinicians, even if quite different from typical hypodermic needles.

Small needle: That needle or other penetrator in a NISE or CISE device and method for use that first penetrates a vessel.

Cannula: That part of a NISE or CISE that enters a vessel after the small needle. It will normally have more than one cutting edge, integrated into a single movable unit, but it may possibly consist of multiple, independently moving parts. (Note: This definition is specific to the NISE or CISE; cannula frequently has other meanings elsewhere.) In certain cases, the cannula might be absent from a NISE or CISE device.

Access needle: A catheter, a hollow needle similar in shape to a typical hypodermic needle, or another penetrator that is typically large relative to the vessel, but is always the final penetrator, and which always remains in the vessel during the treatment or other procedure for which the vessel penetration is made.

Means: A means of doing or implementing an action, feature, and so on, known or obvious to those skilled in the art.

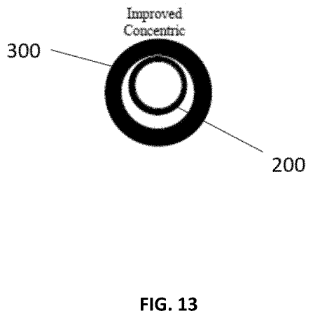

Improved Concentric: An assembly of a nested tubular structures with the innermost tubular structure having a center point that is directionally aligned with a center point of the larger structure; this assembly is shown in FIG. 13.

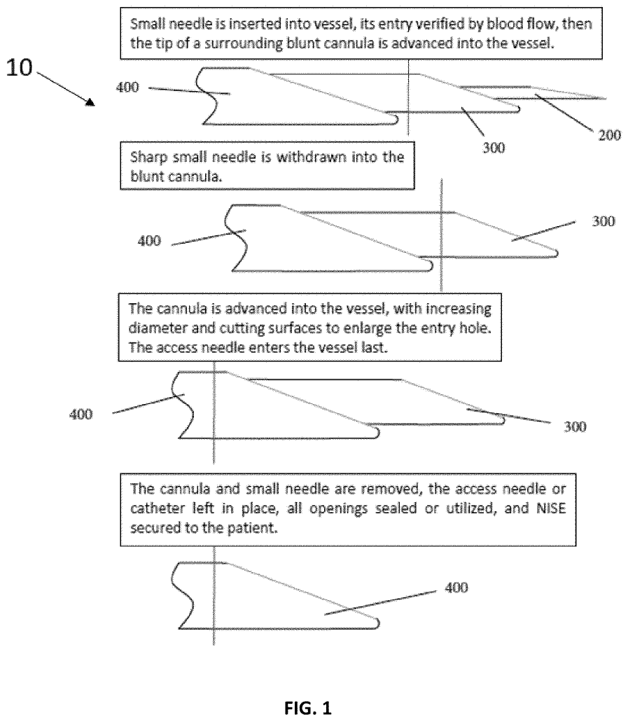

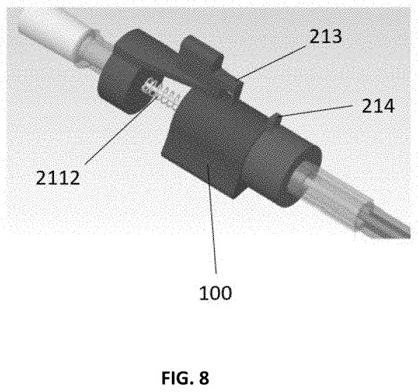

Referring now to FIGS. 1-13, various views of a large needle device configured for insertion into a vessel and a method for use of the device in providing a large needle insertion procedure that is safer and easier, generally referred to herein as the NISE or CISE device 10. Referring now specifically to FIG. 1, a schematic of the basic stages of the exemplary NISE or CISE device 10 showing insertion within the vessel from top to bottom relative to FIG. 1 is shown. The line represents the insertion site of the vessel wall, without regard to insertion angle or necessary change in insertion angle as insertion progresses. The parts are not to scale with respect to one another or exact relative position. The views are cutaway at the center axis or provided in a side view. First, a sharp penetrator indicated herein as small needle 200 is inserted into the vessel to form an entry hole. Although the schematic depicts normal syringe-needle-shaped points, many penetrator, needle, and needle point shapes are possible as suitable means to first penetrate the vessel, depending on the needs of the specific clinical situation; this applies to all penetrators. Normally, the operator will then verify that the small needle 200 has entered the vessel by the presence of blood emitted or visible from the small needle 200. Normally also, once penetration has been verified, the operator then advances a tip of a surrounding blunt cannula 300 into the vessel, preferably only far enough that resistance to penetration is no longer increasing.

Next the sharp penetrator small needle 200 is retracted while continuing to hold the NISE or CISE device 10 in position within the vessel. The retraction may be by manual action, a released spring, or other suitable means. Certain clinical situations may possibly make retraction at other times more appropriate.

The surrounding blunt cannula 300 is then advanced into the vessel all the way (if possible) to enlarge the diameter of the entry hole created by the sharp penetrator small needle 200. This blunt cannula 300 continues penetration through the tip. Surrounding the cannula 300 is an access needle 400, which is inserted into the vessel last. If the cannula 300 could not be advanced fully during use, then an operator of the device 10 would hold the cannula 300 in position and the access needle 400 is advanced over it and into the target vessel.

When the cannula 300 and access needle 400 are inserted fully into the vessel and the access needle 400 is fully advanced, then the access needle 400 is held in position and the cannula 300 and small needle 200 are removed.

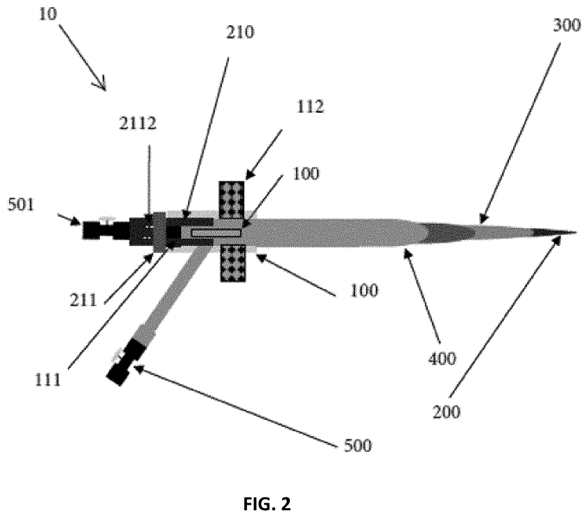

Referring now specifically to FIG. 2, a first embodiment of an exemplary NISE or CISE device 10 is shown and generally described in means functionality to disclose the minimal required features and working portions of the device 10. The NISE or CISE device 10 at a minimum includes a body portion 100. The body portion 100 generally forming a structure for the working components of the NISE or CISE device 10 and functions as a chassis, or other means to hold the different parts together and in a proper aligned relationship relative to one another. The body portion 100 may be a stand-alone structure or it may also be integrated into any component of the NISE or CISE device 10. One of the more suggested assemblies of the body portion 100 of the NISE or CISE device 10 would be to utilize fluid tubing for the main structural components of the body portion 100.

The large access needle 400 is generally the largest needle of the device 10 and is generally positioned as the outermost penetrator of the device 10. The large access needle 400 is generally a hollow structure, such as, but not limited to a catheter, and functions as a hollow needle or other means to conduct blood or other fluid to or from the patient. If the NISE or CISE device 10 is provided without the cannula 300, the large access needle 400 must have a dull tip.

The NISE or CISE device 10 includes a port 500. The port 500 being a connection means to conduct fluid from or to the NISE or CISE device 10. The port 500 including a means to stop or control fluid flow, such as a valve 501.

Accordingly, the NISE or CISE device 10 includes a suitable passageway within the body portion 100 or means for providing a suitable passageway for movement of fluid from the larger access needle 400 to the port 500.

The cannula 300 is positioned internal to the large needle 400 and is normally expected to provide one or more means of continuing to enlarge the hole in the vessel by the presentation of a first cutting edge that is followed by a second cutting edge being a successive cutting edge to the vessel, wherein this generally stepped or tapered cannula shape with the surrounding edge and successive cutting edge encouraging expansion of the vessel during insertion and with minimal trauma to surrounding tissues and the vessel.

The small needle 200 or other suitable penetrator means is relatively the smallest diameter penetrator of the NISE or CISE device 10 and is required to make the first penetration into the target vessel on a trial basis; the small needle 200 means must cause only minimal trauma if misdirected. The small needle 200 is generally received within an interior space of the cannula 300 in a retractable fashion, wherein the small needle 200 is movable from a position within an interior of the cannula 300 to an exterior position distal to the cannula 300 tip when needed. Accordingly, the cannula 300 has the additional function of providing a protective shield around the small needle 200 until it is advanced to a position exterior the cannula 300 for puncturing the target vessel. In the depicted embodiment (FIG. 2), the small needle 200 is positioned on an actuated portion having flexible wings 210. Accordingly, movement of the small needle 200 is articulated to the flexible wings 210, wherein the flexible wings 210 are movable to lock the small needle 200 into an extended position for penetration and the prevention of rotation of the small needle 200 during use. Release of the flexible wings 210 from the locked position will retract the small needle 200 to a protected position within the cannula 300. Further, the flexible wings 210 along with a handle 211 connected to the removable cannula 300, function as a means to maintain all the needle/penetrators 200, 300, 400 in the correct orientation and coaxial alignment relative to one another and within the relative interior spaces for retraction and extension as needed.

Accordingly, to ensure proper function of the NISE or CISE device 10, the small needle 200 is generally received on a means to enable slidable movement, wherein the small needle 200 is movable along the slidable means to advance the small needle 200 past the distal portion of the cannula 300 that follows it and into an extended position for penetrating the target vessel. This means for slidable movement is configured to correspondingly retract the small needle 200 so that it is received completely inside the following cannula 300 so as to protect the vessel from the small needle 200 during additional advancement into the vessel. Preferably this slidable means would be automatic through the use of a spring 2112 to bias the small needle 200 when the operator directs it, wherein the small needle 200 is retained inside the cannula 300 without further operator attention to it.

Upon proper penetration of the NISE or CISE device 10 small needle 200 into the target vessel, a means is provided for the operator to detect blood flow from the vessel into the small needle 200 so as to inform the operator of correct initial placement or otherwise. Preferably, this means is provided through the valve 501 in communication with the fluid path suitable pathway of the body portion 100.

The NISE or CISE device 10 cooperating parts and assembly structures should allow the operator to sense the insertion force of suitable penetrators at all times. If this is not inherent in this preferred configuration, suitable means, such as, but not limited to, a compression, flexible, or resilient member should be provided. In the depicted embodiment, this means to sense insertion force is an elastomeric plug 111. Still further, this suitable means to sense insertion force, may also have additional functions to seal blood emission from the small needle 200 to the exterior of the NISE or CISE device 10, preferably without operator action.

As fluid control is paramount to the NISE or CISE device 10, a means, such as the valve 501 and port 500 is provided to prevent or minimize blood flow to the outside the NISE or CISE device 10 during placement.

As was mentioned previously in regard to the insertion force, a means is provided to prevent or limit blood or other fluid flow to outside the NISE or CISE device 10 specifically during insertion and as the cannula 300 and small needle 200 are removed from the vessel. This means is configured to prevent such flow during the treatment or other procedure, often at considerable pressure (up to 250 mm Hg and -250 mm Hg during dialysis, for example). Specifically, within this FIG. 2 depicted exemplary device, the elastomeric plug 111 is utilized as the means to limit and prevent blood flow to the exterior of the NISE or CISE device 10 during use.

During the time that small needle 200 or cannula 300 are being inserted, it is desirable to limit the force against the vessel or other tissue. A means should be provided to do this to prevent either tissue or NISE or CISE device 10 damage and allow the operator to assess the situation and take corrective or alternative action. In this preferred exemplary assembly of FIG. 2, this function is provided by defined friction between the cannula outermost surface and the elastomeric plug 111.

To comport with the goals and use of the NISE or CISE device 10, all NISE or CISE device 10 surfaces in contact with blood or IV fluid or in or near the penetration site must be sterile; where needed, suitable means must be provided to enable needed sterilization. Optionally, a means should be provided so that the NISE or CISE device 10 may be successfully inserted with one hand at all insertion stages and be able to be held with one hand while the other hand may be utilized to secure the NISE or CISE device 10 to the patient, such as use of the tape down wings 112. Optionally, the above means, features, and concepts may be realized in such fashion that existing off-the-shelf vascular access devices may be incorporated into the NISE or CISE device 10 with minimal modification to the existing device. Note that the various embodiments of the NISE or CISE device 10 and its features depicted schematically in FIG. 2 are not to be construed as the limiting the possible means by which the function of that embodiment may be implemented and thus be covered by this patent. Also, the conceptualized NISE or CISE device 10 presented schematically in FIG. 2 does not apply completely to any of the embodiments described in paragraphs below.

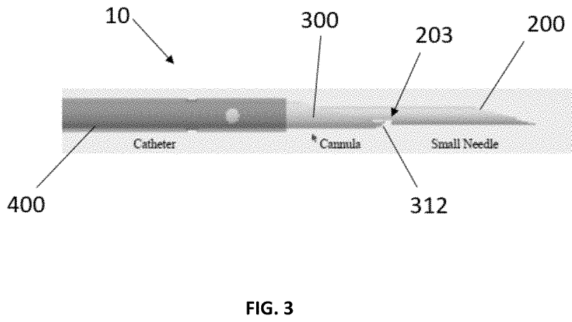

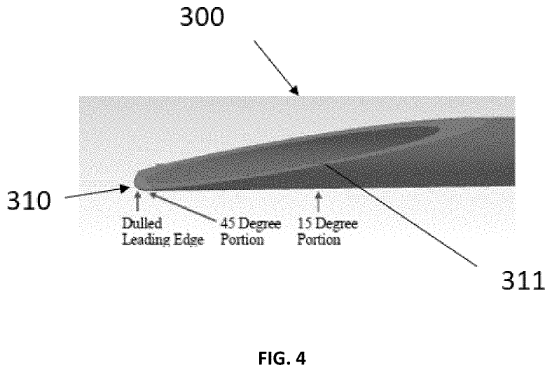

Referring now generally to FIGS. 3-6 in exemplary views of the CISE device 10 of the present disclosure, in FIG. 4 the cannula 300 has a tip 310 functioning as a penetrator with the tip 310 culminating in a dulled leading edge having a specific grind at a 45 degree angle transitioning to a slope of the traditional 15 degree normal grind of a surrounding edge 311 of the cannula 300. Accordingly, the tip 310 of the cannula 300 is dulled or blunt and the other surrounding edges 311 are sharp and capable of cutting tissue. Although the dullness of this cannula 300 tip 310 is preferred to prevent vessel damage during insertion, it does increase the insertion force necessary to properly seat the device 10.

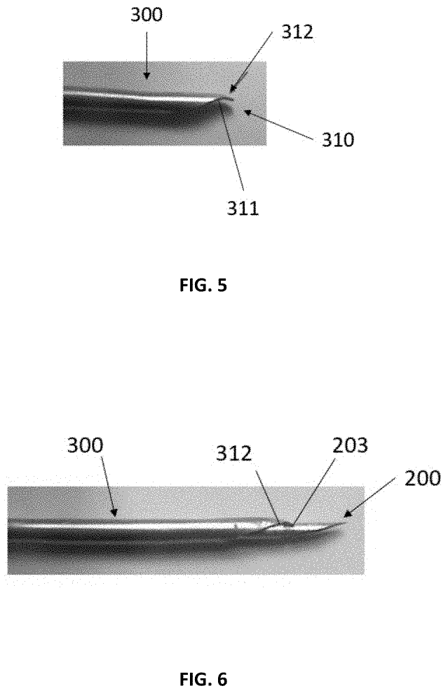

Therefore, as is shown in FIGS. 3, 5-6, to mitigate this insertion force, the CISE device 10 cannula tip 310 is provided with a curved shaped generally orientated inwards relative to the interior to form a beak 312. This beak 312 is utilized for nesting within a corresponding slot 203 positioned on the small needle 200. This slot 203 allows for the beak 312 to be seated in a recessed position, wherein this recessed position functions as a mitigation to the insertion force of a blunt cannula tip. Accordingly, as is shown in FIG. 6, the slot 203 of the small needle 200 hides the beak 312 of the cannula 300 during insertion. As is seen in FIG. 5, when the small needle 200 of the CISE device 10 is retracted, only the dull cannula tip 310 remains in the vessel. The angular portion of the cannula surrounding edge 311 at its 15 degree slope remains sharp and may continue to cut into the vessel during further insertion. When the beak 312 is used, as barely visible in FIG. 5, the 45 degree grind has the opposite slope to that shown in FIG. 4, this, the slope is opposite that of the main 15 degree grind. This geometry makes the beak 312 more effective by helping to retain the beak 312 in the slot 203 and reduces the needed blunt area of the bend of the beak 312.

Additionally, as the CISE device 10 preferably utilizes the spring 2112, the beak 312 when received in the slot 203 provides an interference against retraction of the small needle 200. This assembly enables the beak 312 of the cannula 300 to hold the small needle 200 out from the cannula 300 tip 310 against both the force of the spring 2112 and insertion force on the small needle 200 during insertion of the CISE device 10.

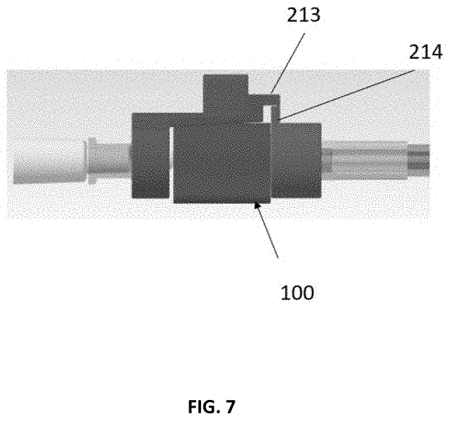

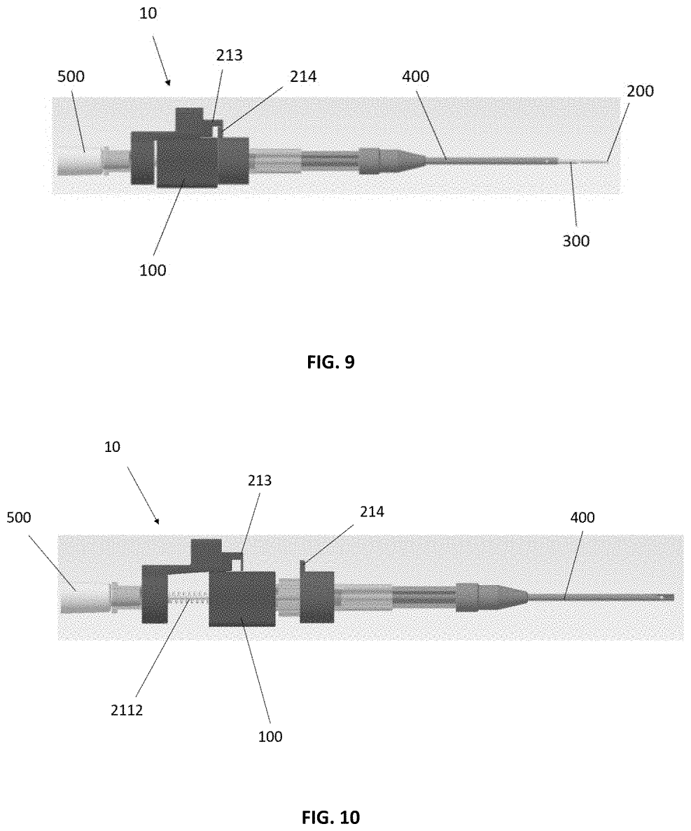

Referring now to FIGS. 7-10 of the CISE device 10, to separate the interference of the beak 312 when nested within the slot 203 of the small needle 200, the actuated portion flexible wing assembly 210 is replaced with a rotating mechanism. This rotating mechanism utilizes a lever 213 coupled to a retaining post 214 on the body 100, wherein the sufficient flexibility of the small needle 200 and cannula 300 allow for a slight rotational movement of the lever 213 relative to the retaining post 214 to activate the spring 2112. Accordingly, upon disengagement of the lever 213 with the retaining post 214 the needle 200 is retracted to a position within the cannula 300 interior. Correspondingly, advancement of the needle 200 to an exterior position of the cannula 300 will advance the needle slot 203 into engagement with the beak 312 and allow for seating the lever 213 upon the retaining post 214 for temporarily fixing the position of the needle 200 exterior to the cannula 300 tip 310. In the preferred assembly of the CISE device 10, as is shown in FIG. 9-10, an experimental device is constructed using a stock ClampCath needle assembly having an approximately 17 gauge (0.057 inch Outer Diameter (“O.D.”), 0.040 inch Inner Diameter (“I.D.”)) needle functioning as the cannula 300 and placed within the catheter 400. This CISE device 10 needle (cannula 300) is modified as above with the beak 312 and 45 degree back grind. The small needle 200 within this exemplary CISE device 10 is a 20 gauge (0.036 inch O.D., 0.025 inch I.D.) standard needle.

As is shown in FIG. 9, during engagement of the lever 213 with the retaining post 214 the cannula 300 is generally constrained from retraction during insertion into a target vessel. If an obstruction would happen to be encountered during this insertion process, the cannula 300 can be allowed to be released or free to move by disengagement of the lever 213 from the retaining post 214 as is shown in FIG. 10. Accordingly, the release of the constraint upon of the cannula 300 can occur simultaneously with the retraction of the small needle 200. In FIG. 10, a holder of the cannula 300 is no longer constrained between a holder of the small needle 200 and a retainer. Therefore, the cannula 300 may be allowed to move within the CISE device 10 relative to an encountered obstruction or excessive force upon the cannula 300 during insertion. Although depicted as restrained by the small needle 200 lever 213 and post 214, the cannula 300 may be provided in a movable assembly upon encountering obstruction that is not controlled by the small needle 200 for particular use cases.

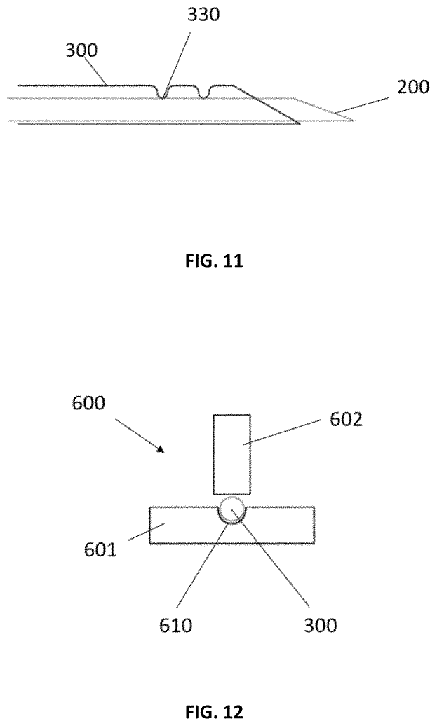

As is shown in FIG. 5, the canula beak 312 presents a curved forward facing surface that is blunt and may impede a smooth insertion. To generally minimize the insertion force of this beak 312, a smaller beak or effectively smaller beak is preferred. Accordingly, applicants have effectively created a smaller beak for the CISE device 10 by utilizing the improved concentric assembly shown diagrammatically in FIG. 13 through the placement of a crimp 330 in the cannula 300 body side opposite the leading edge or point of the small needle 200 as can be seen in FIG. 11.

Referring now to FIGS. 11-12, a pair of crimps 330 is shown in the cannula body side 300 with the crimps 330 having a depth selected to generally reduce the size of the cannula 300 interior to a size approximating the exterior diameter of the small needle 200 and a placement to generally align the cannula 300 tip 310 and small needle 200 point. These crimps 330 effectively provide the exemplary improved concentric configuration of FIG. 13. In a preferred embodiment of the present disclosure, the CISE device 10 utilizes a pair of crimps 330 placed 1 cm apart. An exemplary method of assembly for the crimp 330 within the cannula 300 body portion is shown in FIG. 12 wherein a jig 600 is utilized to ensure proper seating and depth of the crimp 330. Accordingly, this jig 600 utilizes a base block 601 having a round groove 610 to retain an opposite side of the tubing (cannula 300) while a ram 602 on a precision press provides a downward force to contact the tubing and mechanically deform the tubing to form the crimp 330. The round groove 610 is provided in a shape to correspond to the tubing (cannula 300) outside diameter and secures the point side within the groove 610 to retain the shape of the tubing. The ram 602 having a shape corresponding to a size of the desired crimp 330.

In a detailed method or procedure for use of the jig 600 an automated or manual process may utilize the following steps. First, the smaller tubing 200 intended to be improved concentrically aligned with the larger tubing 300 is placed within the larger tubing 300. This assembly of the smaller tubing 200 within the larger tubing 300 is placed in the proper orientation in the round groove 610. The proper orientation is to align the tubing 200, 300 relative to their points with the points placed in adjacency and nearest the round groove 610 opposite the ram 602. The ram 602 is then activated to crimp the larger tube 300 until the larger tube 300 is deformed to the point where it contacts the smaller tube 200 with light friction detected by force to move the smaller tube 200 back and forth within the larger tube 300. The ram 602 is then slightly raised and the smaller tube 200 is retracted from within the larger tube 300. The ram 602 is then further depressed to a contact point to accommodate a yield point of the larger tubing 300. This may be approximately 0.004 of an inch, depending upon the material properties and size of the outer tubing 300. Alternately, the ram 602 may be provided in a multitude of sizes and geometries depending upon the constraint needs of the smaller tubing 200 within the larger tubing 300, provided that the smaller tubing 200 is constrained both top to bottom and left to right relative to its nested receipt within the larger tubing 300.

During the injection process of the CISE device 10 a seal between the cannula 300 and access needle 400 (or elsewhere in the access device) prevents or minimizes blood leakage. An additional seal may also be provided between the small needle 200 and the cannula 300. Additional means may also be provided to achieve a complete seal after the cannula 300 and small needle 200 have been removed.

The cannula 300 has both dulled and sharp geometries and a generally enlarging diameter as it passes into the vessel. There are many suitable means to make this penetration and dilation, depending on various clinical situations, which means are known or obvious to those skilled in the art. The cannula 300 may easily be created by stainless steel needle stock in successive layers around the sharp penetrator needle, welded or glued to each other. Although the schematic of FIG.1 shows that this next stage uses a dulled needle, in actual fact, this and any succeeding stages of the cannula 300 may be constructed of sharp needles. As above, many penetrator shapes, with different dulled and sharp geometries, may be a suitable means to make these create the cannula 300. The NISE or CISE device 10 may incorporate a means to secure the NISE device to the patient, such as the tape down wings 112 to avoid vessel trauma from movement or the NISE or CISE device 10 being dislodged from the vessel. If this means to secure is provided, it should be used just before the time when all interior objects, such as the small needle 200 and cannula 300 are withdrawn and the resultant opening either sealed by suitable means or utilized for blood or other fluid transfer.

As penetration into or within a vessel by a penetrator creates forces in both a vertical and horizontal direction, the hole created within the tissue by the penetrator is often considered to be a shape that can be described as slightly oblong. Therefore, the bevel-up positional orientation seen in FIG. 3 of the NISE or CISE device 10 attempts to place any successive cutting portions or points into the largest portion of the initial hole. Accordingly, this NISE or CISE device 10 positional orientation enables a smoother advance into the vessel. Because the hole is formed due to the elasticity of the vessel, its shape is independent of prior needle shape or orientation, to a first approximation.

In another aspect of the present disclosure, the NISE or CISE device 10 cannula 300 might not be included, or if included, the number of successive cutting edges (or stages) of the cannula 300 may be other than two. A brief explanation is in order as these variations are within the scope of this disclosure. Clinical needs or situations may arise where a difference between an outer diameter of the small needle 200 and an inner diameter of the access needle 400 may be too small to economically, or even practicably, allow room for the two-stage blunt cannula 300 depicted in FIG. 2. There might only be room for a single-stage blunt cannula 300, or even none at all. The retractable small needle concept is still useful in such a case. Or, on the other hand, a large size difference may even mandate three or more cutting stages in the blunt cannula 300.

In another embodiment of the present disclosure, the NISE or CISE device 10 features are incorporated to an existing and already available vascular device by augmenting an off-the-shelf vascular access device by converting it into a NISE device 10.

Accordingly, even simple medical devices and accessories entail high regulatory compliance and approval costs. Once in production, each step of the production process, each part, and the production facility itself, all separately and together entail both high regulatory compliance costs and capital investment. For these same reasons, changes to an existing device or even modifications to production workflow are also expensive. Therefore, it may be desirable to add NISE or CISE device 10 components to an existing vascular access device to leverage this existing investment in facilities, training, regulatory, and other costs, while at the same time minimizing changes to the existing device.

The device and method of the present disclosure may be used in combination with an existing vascular access device, wherein the NISE or CISE device 10 add-on will advantageously render the existing vascular access device three significant novel features as discussed in the above paragraph [0011]. The basic operating principles resultant in the NISE or CISE device 10 when incorporated into existing vascular devices will be virtually the same as disclosed herein.

Still further, an optional modification to the stock device may be carried out during the incorporation of already available vascular devices: dulling the point of the off-the-shelf device could minimize vessel trauma if the device is improperly inserted or perturbed.

Any add-on parts and integrated off-the-shelf devices when mated together to form a complete NISE or CISE device 10 must be sterilized. For typical needle assemblies, the fluid path, the needle, and (presumably) the needle cap are sterilized. If either gamma or electron beam irradiation are suitable and utilized, there are no further issues. But there are two potential problem areas if gas is used (e.g., ethylene oxide or steam); the interface between the outer tube and the outlet tube, and the hole created by the cannula.

If the device is merely capped with the access needle cap, sterilant gas can be pumped into the inlet. It will flow through the cap, through and alongside the added penetrators such as the small needle and cannula, then out. That will almost accomplish the sterilization of the fluid path, access needle, and cap. Also, when the large tube is rotated or moved axially, its wall would not have been sterilized, so it will result in another small non-sterile place in the fluid path.

If gamma or electron beam sterilization cannot be used, both of these issues must be addressed. With respect to the large and small tubes, one possibility would be to install the outlet port, clamp, and hanger on the outlet tube, and dip the end of the tube in 8% H₂O₂. This concentration of hydrogen peroxide is compatible with PVC and silicone, is not hazardous, and is FDA approved as a liquid sterilant if in the H₂O₂ for six hours. So, a batch of tube ends and large tube segments might be left immersed overnight, as well as a long billed instrument such as a hemostat. The blood tubes are then simply pulled through the small tubes, while still under the H₂O₂. Because the tube tension will squeeze out virtually all of the H₂O₂ (harmless in tiny amounts), the problem surface is sealed off before exposure to atmosphere and water rinsing is not needed. Then the interior of the blood tubes are shaken out, dried, and adhered to the body of the access device as per normal manufacturing of the devices.

As best understood by the time of this disclosure, It may be economical to sterilize any small spaces withing the device with a narrow electron beam, if a completely satisfactory solution to sterilizing the entire surface with gas sterilant is lacking. Gas sterilization would then follow. These solutions are admittedly somewhat unwieldy; gamma sterilization would therefore seem to be the best solution for this configuration of the first embodiment of the NISE or CISE device 10 incorporated into a an existing fistula needle.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A method of making large needle insertion into an artery or vein easier and with less unnecessary trauma to the blood vessel, the method comprising the steps of:
providing a large needle comprising a first penetrator and a second penetrator, the first
penetrator comprising a sharp point and the second penetrator comprising a blunted point.

2. The method as in claim 1, wherein the first penetrator has a smaller diameter than the second penetrator.

3. The method as in claim 2, further comprising the steps of:
providing a cannula in the large needle between the first penetrator and the second penetrator;
inserting the first penetrator (sharp needle) into a target vessel;
retracting the first penetrator into the cannula;
carrying the cannula into the target vessel;
carrying the second penetrator of the large needle into the target vessel; and
removing the cannula containing the retracted first penetrator from the large needle.

4. The method as in claim 3, wherein the cannula has a cylindrical shape and comprises increasing diameters and a cutting outer surface with a beak.

5. The method as in claim 4, wherein the first penetrator includes an slot, the slot sized to receive the beak in an extended position.

6. A device for facilitating large needle insertion into a vessel, the device comprising:
a second penetrator, the second penetrator being a large tube, such as a catheter and comprising blunt points;
a cannula, the cannula received within an interior of the second penetrator and comprising a dull tip with a beak, increasing diameter, and an outer cutting surface; and
a first penetrator, the first penetrator slidably received within an interior of the cannula, the first penetrator having a sharp point.

7. The device as in claim 6, wherein the first penetrator includes a slot, the slot sized to receive the beak, wherein the receipt of the beak within the slot restrains the movement of the first penetrator relative to its position within the cannula.

8. The device as in claim 7, wherein the cannula includes at least one crimp, the at least one crimp being a deformation of an outer wall of the cannula towards the interior space and opposed the sharp point of the received first penetrator, wherein the at least one crimp reduces a portion of the cannula inner diameter to place a tip of the cannula and the sharp point of the first penetrator in an improved concentric alignment.

9. The device as in claim 8, wherein the cannula includes a pair of crimps.

10. The device as in claim 6, wherein a body portion of the device includes a post for receiving a lever on an end of the first penetrator opposed the sharp point, wherein rotational engagement of the lever with the post secures to position of the first penetrator exterior to a tip of the cannula.

11. A method of manufacturing the device of claim 8, the method comprising the steps of:
placing the first penetrator within the interior of the cannula with pointed end of the first penetrator aligned with the tip of the cannula;
placing the nested assembly of the first penetrator within the cannula into a base block having a round groove corresponding to a size of an outer diameter of the cannula;
depressing a ram to apply downward pressure on an exterior of the cannula towards the base block util the deformed cannula wall makes contact with the first penetrator and friction is detected by force to move the small penetrator back and forth relative to the cannula;
raising the ram slightly to decrease the downward pressure;
retracting the first penetrator from the nested receipt within the cannula;
further depressing the ram past the initial point of contact with the first penetrator to accommodate a yield point of the cannula; and
ensuring that nested receipt of the first penetrator within the cannula interior results in sliding friction.

12. A device to aid in a large needle insertion into a vessel, the device comprising:
a body portion; the body portion defining a fluid pathway;
the large needle received on the body portion at a fixed position and in communication with the fluid pathway;
a cannula, the cannula coupled to a means to move the cannula coaxially within an interior of the large needle to an extended position at least exterior to an end of the large needle;
a small needle, the small needle having a sharp point and coupled to means to move the small needle coaxially within an interior of cannula to an extended position at least exterior to an end of the cannula, wherein the small needle is nestedly received within the cannula and the cannula is nestedly received within the large needle for at least a portion of its movement;
a means to fix the small needle in the extended position;
a means to retract the small needle from the extended position to a protected position within the cannula interior;
a means to seal the small needle, cannula, and large needle from fluid leakage relative to each other and the body portion; and
a port, the port received on the body and in fluid communication with the large needle, the port including a means to stop or control fluid within the fluid pathway.

13. The device as in claim 12, wherein the cannula has a dulled point and a beak.

14. The device as in claim 13, wherein the small needle includes a slot, the slot aligned with the beak and having a size to accommodate the beak.

15. The device as in claim 14, wherein the receipt of the beak within the slot is at least a portion of the means to fix the small needle in the extend position.

16. The device as in claim 14, wherein a body portion includes a post for receiving a lever on an end of the small needle opposed the sharp point, wherein rotational engagement of the lever with the post is a portion of the means to fix the small needle in the extended position.

17. The device as in claim 12, wherein the cannula includes at least one crimp, the at least one crimp being a deformation of an outer wall of the cannula towards the interior space and opposed the sharp point of the received small needle, wherein the at least one crimp reduces a portion of the cannula inner diameter to place a tip of the cannula and the sharp point of the small needle in an improved concentric alignment.

18. The device as in claim 17, wherein the cannula includes a pair of crimps.

19. A method of manufacturing the device of claim 17, the method comprising the steps of:
placing the small needle within the interior of the cannula with pointed end of the small needle aligned with the tip of the cannula;
placing the nested assembly of the small needle within the cannula into a base block having a round groove corresponding to a size of an outer diameter of the cannula;
depressing a ram to apply downward pressure on an exterior of the cannula towards the base block util the deformed cannula wall makes contact with the small needle and friction is detected by force to move the small penetrator back and forth relative to the cannula;
raising the ram slightly to decrease the downward pressure;
retracting the small needle from the nested receipt within the cannula;
further depressing the ram past the initial point of contact with the small needle to accommodate a yield point of the cannula; and
ensuring that nested receipt of the small needle within the cannula interior results in sliding friction.

20. The device as in claim 12, wherein the device includes a means to enable correct orientation of the small needle, cannula, and large needle relative to each other and includes a means to detect a flow of blood within the device to ensure proper placement and penetration of the small needle.
